(19) United States
(12) Patent Application Publication
(10) Pub. No.: US 2008/0182312 A1
(43) Pub. Date: Jul. 31, 2008

(54) Stable reagents and kits useful in loop-mediated isothermal amplification (LAMP)

(76) Inventors: Todd Denison Pack, Cincinnati, OH (US); Xiaokang Deng, Cincinnati, OH (US)

Correspondence Address: MORRISON & FOERSTER LLP, 12531 HIGH BLUFF DRIVE, SUITE 100, SAN DIEGO, CA, 92130-2040, US

(21) Appl. No.: 12/009,183

(22) Filed: Jan. 16, 2008

Related U.S. Application Data
(60) Provisional application No. 60/880,988, filed on Jan. 17, 2007.

(51) Int. Cl. C12N 9/00

(52) U.S. Cl. 435/183

(57) ABSTRACT

Provided herein is a reagent preparation for loop-mediated isothermal amplification of nucleic acids comprising: at least one polymerase enzyme, a target-specific primer set, and dinucleotide triphosphates (dNTPs) in a single, dry format; wherein said reagent preparation is water soluble and stable above 4° C.

### CROSS-REFERENCE TO RELATED APPLICATIONS

This application claims priority to provisional U.S. Patent Application Ser. No. 60/880,988, filed Jan. 17, 2007, the content of which is hereby incorporated by reference in its entirety.

### TECHNICAL FIELD

The invention relates to the long-term storage of biological materials and reagents useful in nucleic acid amplification. In particular, it relates to dry compositions of biological reagents necessary for loop-mediated isothermal amplification (LAMP) of nucleic acids and methods of making such compositions.

### BACKGROUND ART

Point-of-care diagnostic devices permit physicians to obtain rapid, inexpensive information crucial to providing effective patient care. For diagnosis of an infectious disease, gene amplification devices theoretically can provide rapid and sensitive identification while eliminating the need for pathogen cultures and/or large biological sample size. A rapid, specific genetic amplification device also permits the detection of specific alleles or other genetic risk factors that facilitate individualized tailoring of therapeutic regimens. Methods for gene amplification include polymerase chain reaction (PCR), strand displacement amplification (SDA), ligase chain reaction (LCR), and transcription mediated amplification (TCA). See, e.g., U.S. Pat. Nos. 4,683,195; 4,629,689; 5,427,930; 5,339,491; and 5,409,818. However, these technologies are limited by the number of multiple reagents with varying stability for such amplification as well as a reliance on expensive equipment.

Loop-mediated isothermal amplification (LAMP) overcomes the dependence on expensive equipment (via elimination of thermocycling and the requirement for machine-based result detection) while amplifying DNA rapidly and specifically. Notomi et al., Nucl. Acids Res. 28:E63 (2000); U.S. Pat. No. 6,410,2778. In one example, the method simply incubates a mixture of the target gene, four or six different primers, Bst DNA polymerase, and substrates and results in high specificity amplification under isothermal conditions (60 to 65° C.). The presence of the target DNA is then determined by visual assessment of the turbidity or fluorescence of the reaction mixture, which is kept in the reaction tube. Mori et al., Biochem. Biophys. Res. Commun. 289:150-54 (2001). Because of the advantage in rapid, efficient, and specific amplification of small amounts of DNA, LAMP has emerged as a powerful tool to facilitate genetic testing for the rapid diagnosis of viral and bacterial infectious diseases in clinical laboratories.

However, the usefulness of LAMP in the clinic remains limited by having the individual reagents shipped and stored in a multi-tube format with enzymes stored in glycerol at −20° C. or below. The reagents must be handled and recombined without stray nucleic acid or DNAse/RNAse contamination in order to fully enjoy the sensitivity, specificity and efficiency of LAMP amplification. Typically, the first step in the LAMP method is thawing the multiple tubes of reagents and preparing the master mix. The master mix requires the combining the reagents in the Reaction Mix tube and Primer Mix tube as well as adding water while the master mix is kept on ice. The master mix is then heated at 95° C. for 5 minutes after which it is placed back on ice. The tube is then reopened and the polymerase enzyme, and reverse transcriptase enzyme if required, is added. The master mix is then added to sample tubes along with the sample. The tube is closed and placed at about 65° C. for the LAMP reaction to occur. See FIG. 1 for illustration.

The multiple steps needed for the LAMP reaction preparation procedure would reduce its acceptance in a clinical laboratory setting. In a clinical laboratory setting, ease-of-use is an important factor especially when testing batched, or multiple samples. A procedure that is tedious can lead to increase errors.

In addition to the multiple steps, the storage at −20° C. increases the difficulty in performing the test as the product must be thawed prior to use. Furthermore, the requirement of storage at −20° C. places a burden on the laboratory as freezer space is required.

### SUMMARY OF THE INVENTION

The reagent preparations disclosed herein make the LAMP method accessible and reasonable in virtually any clinical setting. The dry format reagent preparation enhances ease of use, eliminates user error, and provides reagent stability at room temperature. In the dry format, the labile reagents are mixed together in a single container and then dried. Each container holds enough reagents to perform a single reaction. Thus, the user simply adds a reconstitution buffer and a sample, and all the components for the LAMP method are present. The elimination of various combination and thawing steps reduces the likelihood of user error through incorrect handling or contamination. Moreover, in the dry format, the LAMP components are stable if stored at greater than 4° C., eliminating the requirement for freezing during shipping and storage.

More particularly, in one aspect, provided herein is a reagent preparation for loop-mediated isothermal amplification of nucleic acids comprising: at least one polymerase enzyme capable of strand displacement, a target-specific primer set, and dinucleotide triphosphates (dNTPs) in a single, dry format; wherein said reagent preparation is water soluble and stable above 4° C. In some embodiments, the polymerase enzyme is Bst enzyme. If the target is RNA, the reagent preparation also includes a reverse transcriptase enzyme. In some embodiments, the reverse transcriptase is AMV reverse transcriptase.

Further provided herein is a kit comprising the reagent preparation in the disclosed dry format. The kit can further comprise an additional and separate wet format comprising an aqueous buffered solution. In one embodiment, the buffered solution is 25 mM Tris-HCl pH 8.8, 12.5 mM KCl, 10 mM MgSO₄, 12.5 mM (NH₄)₂SO₄, and 0.125% Tween 20.

In another aspect, provided herein is a method of making a reagent preparation for loop-mediated isothermal amplification of nucleic acids comprising the steps of: (a) providing a buffered aqueous solution of (1) at least one polymerase enzyme, wherein the enzyme is capable of strand displacement, (2) a target-specific primer set, (3) dinucleotide triphosphates (dNTPs), wherein said solution is glycerol-free; and (b) drying the solution to form the reagent preparation; wherein the reagent preparation is water soluble and is stable above 4° C.

### BRIEF DESCRIPTION OF THE DRAWINGS

FIG. 1 provides a schematic representation of the loop-mediated isothermal amplification (LAMP) of nucleic acids. FIG. 1a. Generation of the Loopamp Starting Structure. Step 1, forward inner primer region ‘F2’ binds to complementary sequence on the target sequence. The polymerase initiates primer extension while displacing the target complimentary strand. Step 2, polymerase completes copy of target sequence. Step 3, the ‘F3’ primer binds to complementary sequence on the target sequence and polymerase initiates primer extension. Step 4, primer extension from the ‘F3’ primer displaces forward inner primer product. The ‘F1c’ and ‘F1’ on the displaced forward inner primer product hybridize to form a hairpin loop. Step 5, backward inner primer region ‘B2’ binds to complementary sequence on the displaced product. The polymerase initiates primer extension. Step 6, polymerase displaces hairpin and completes primer extension. Step 7, the ‘B3’ primer binds to complementary sequence and primer extension is initiated. Step 8, primer extension completely displaces a single strand product that forms hairpin loops at each end. This is the starting structure for the amplification phase of the Loopamp. Note: Primer extension beginning at the forward inner primer site is shown as a representative initiation of the process—the process can initiate at either the forward inner primer site or backward inner primer site. FIG. 1b. Amplification of Loopamp Starting Structure. Forward inner primer and backward inner primer bind to complementary sequences on the Loopamp starting structure and initiate primer extension and strand displacement by the polymerase. Continued hybridization of the forward inner primer and backward inner primer followed by primer extension and strand displacement results in the formation of product of different lengths and generation of more Loopamp starting structures.

FIG. 2 illustrates the LAMP protocol using a multi-tube wet format for amplification of nucleic acids.

FIG. 3 illustrates the LAMP protocol using a dual tube dry format for amplification of nucleic acids.

### MODES OF CARRYING OUT THE INVENTION

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

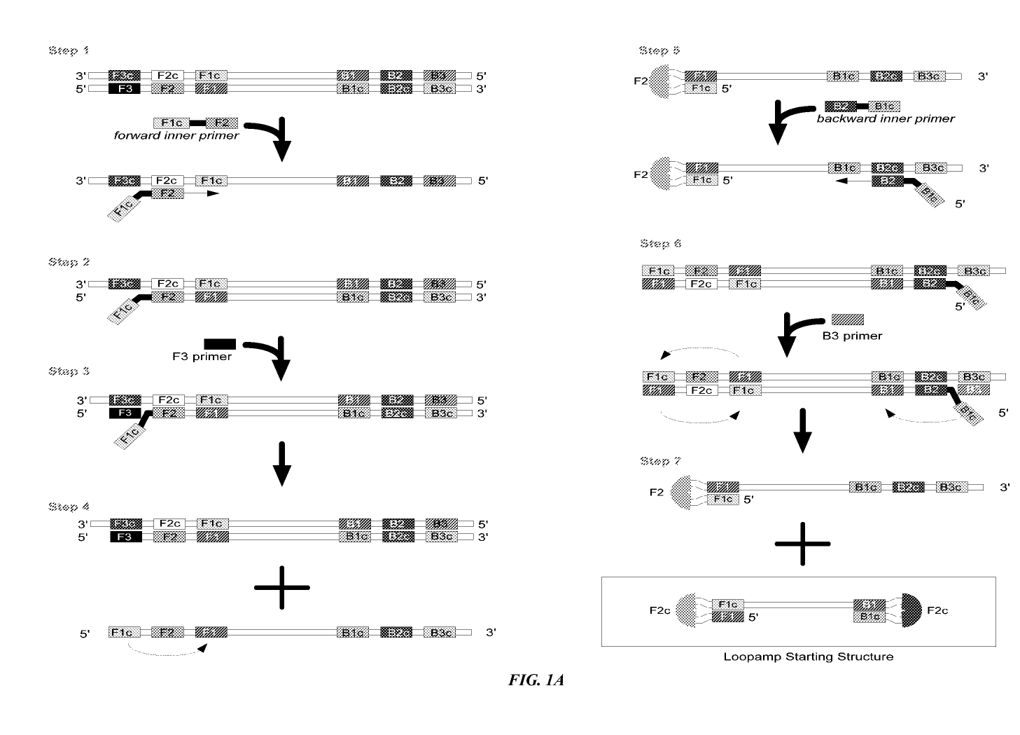

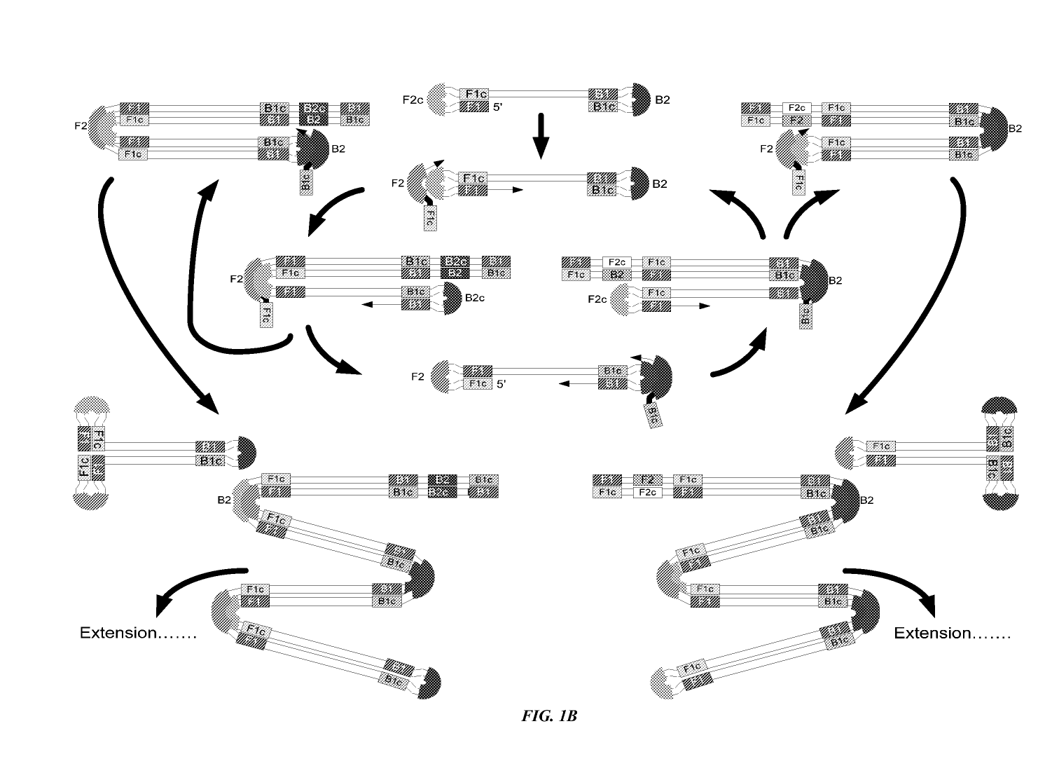

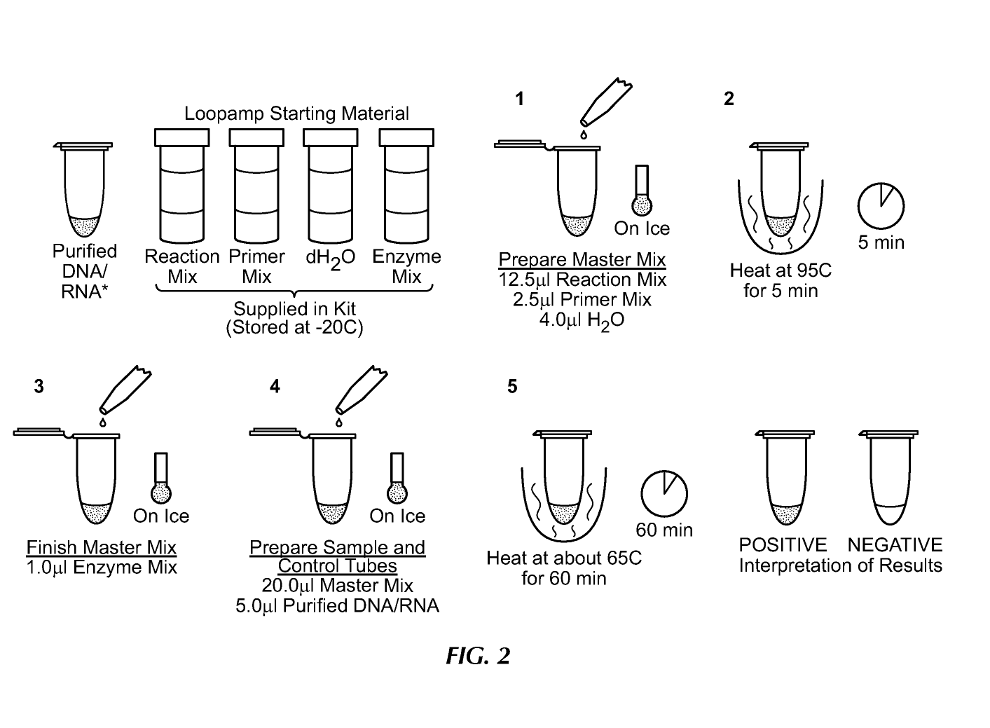

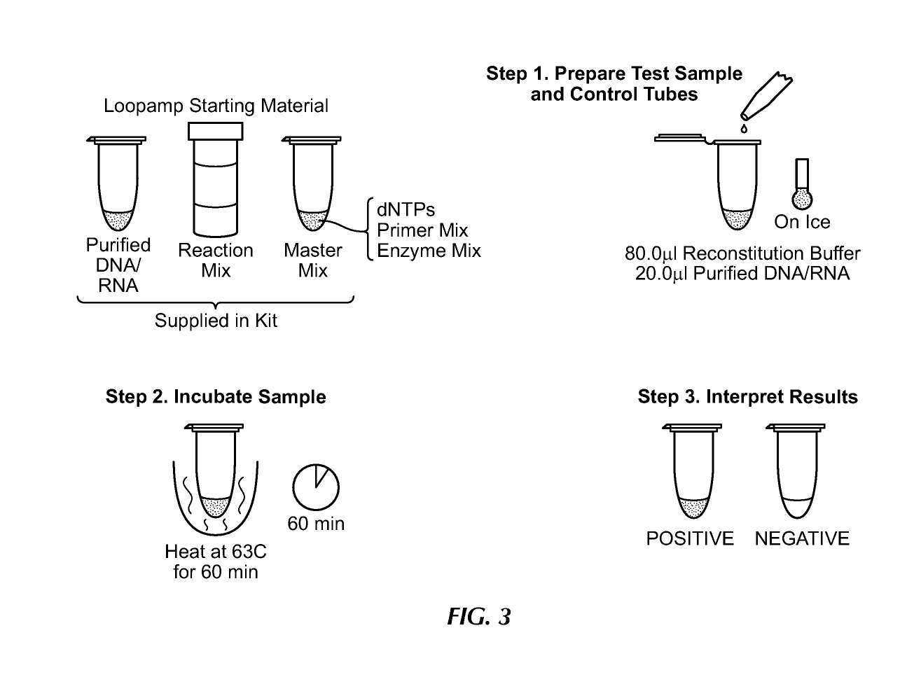

Loop-mediated isothermal amplification (LAMP or Loopamp) is an isothermal DNA amplification procedure using a set of four to six primers, two to three “forward” and two to three “reverse” that specifically recognize the target DNA. See Nagamine et al., Nucleic Acids Res. (2000) 28:e63; Nagamine et al., Clin. Chem. (2001) 47:1742-43; U.S. Pat. No. 6,410,278; U.S. Patent Appl. Nos. 2006/0141452; 2004/0038253; 2003/0207292; and 2003/0129632; and EP Patent Appl. No. 1,231,281. Briefly, one set of primers are designed such that approximately ½ of the primer is positive strand the other ½ of the primer sequence is negative strand. After strand displacement amplification by the polymerase, a nucleic acid structure that has hairpin loops on each side is created. From this structure, repeating rounds of amplification occur, generating various sized product. A by-product of this amplification is the formation of magnesium-pyrophosphate, which forms a white precipitate leading to a turbid reaction solution. This presence of turbidity signifies a positive reaction while the absence of turbidity is a negative reaction. Additional additives, such as calcein, allow other visualizations to occur; as for calcein it enables fluorescence detection. See FIG. 1. The amplification reaction occurs under isothermal conditions (at approximately 65° C.) and continues with an accumulation of 10⁹ copies of target in less than an hour.

In one aspect, provided herein is a reagent preparation for loop-mediated isothermal amplification of nucleic acids comprising: at least one polymerase enzyme, wherein the enzyme is capable of strand displacement, a target-specific primer set, and dinucleotide triphosphates (dNTPs) in a single, dry format; wherein said reagent preparation is water soluble and stable above 4° C. In some embodiments, the polymerase enzyme capable of strand displacement is Bst enzyme. If the target is RNA, the reagent preparation also includes a reverse transcriptase. In some embodiments, the reverse transcriptase is AMV reverse transcriptase.

In another aspect, provided herein is a method of making a reagent preparation for loop-mediated isothermal amplification of nucleic acids comprising the steps of: (a) providing a buffered aqueous solution of (1) at least one polymerase enzyme, (2) a target-specific primer set, (3) dinucleotide triphosphates (dNTPs), wherein said solution is glycerol-free; and (b) drying the solution to form the reagent preparation; wherein the reagent preparation is water soluble and is stable above 4° C. If the target is RNA, the method further includes a reverse transcriptase. In some embodiments, the reverse transcriptase is AMV reverse transcriptase.

Any suitable DNA polymerase capable of strand displacement can be employed. As used herein, the term “strand displacement” refers to the ability of the enzyme to separate the DNA strands in a double-stranded DNA molecule during primer-initiated synthesis. The enzyme can be a complete enzyme or a biologically active fragment thereof. The enzyme can be isolated and purified or recombinant. In some embodiments, the enzyme is thermostable. Such an enzyme is stable at elevated temperatures (>40° C.) and heat resistant to the extent that it effectively polymerizes DNA at the temperature employed. Sometimes the enzyme can be only the active portion of the polymerase molecule, e.g., Bst large fragment. Exemplary polymerases include, but are not limited to Bst DNA polymerase, Vent DNA polymerase, Vent (exo-) DNA polymerase, Deep Vent DNA polymerase, Deep Vent (exo-) DNA polymerase, Bca (exo-) DNA polymerase, DNA polymerase I Klenow fragment, Φ29 phage DNA polymerase, Z-Taq™ DNA polymerase, ThermoPhi polymerase, 9°Nm DNA polymerase, and KOD DNA polymerase. See, e.g., U.S. Pat. Nos. 5,814,506; 5,210,036; 5,500,363; 5,352,778; and 5,834,285; Nishioka, M., et al. (2001) J. Biotechnol. 88, 141; Takagi, M., et al. (1997) Appl. Environ. Microbiol. 63, 4504.

If the target nucleotide is RNA, any suitable reverse transcriptase may be employed. In some embodiments, the reverse transcriptase is thermostable. Exemplary examples of reverse transcriptases used to convert an RNA target to DNA include, but are not limited to Avian Myeloblastosis Virus (AMV) reverse transcriptase, Moloney Murine Leukemia Virus (M-MuLV, MMLV, M-MLV) reverse transcriptase, MonsterScript reverse transcriptase, AffinityScript reverse transcriptase, Accuscript reverse transcriptase, StrataScript 5.0 reverse transcriptase 5.0, ImProm-II reverse transcriptase, Thermoscript reverse transcriptase and Thermo-X reverse transcriptase and any genetically altered forms or variants of the aforementioned reverse transcriptases.

The buffered aqueous solution suitable for the compositions and methods provided herein are those that permit the desired activity of the nucleic acid synthesizing enzyme but do not contain glycerol. Glycerol is typically a component of buffered aqueous solutions for enzymes and acts as a stabilizing agent. The presence of glycerol prevents proper drying and thus renders the reagent composition unstable above 4° C. The buffer of the dry and wet format can be the same buffer. The buffer in the wet format can also be the reconstitution buffer. In one embodiment, the aqueous buffer comprises 25 mM Tris-HCl pH 8.8, 12.5 mM KCl, 10 mM MgSO₄, 12.5 mM (NH₄)₂SO₄, and 0.125% Tween 20. In some embodiments, an agent that facilitates melting of the DNA is also included. Exemplary agents that facilitate the melting of DNA include but are not limited to betaine, trehalose, tetramethylone sulfoxide, homoectoine, 2-pyrrolidone, sulfolane, and methyl sulfone.

As used herein, the term “stable” refers to stability of biological activity with less than 20% loss of original activity (as measured after reagents are first dried) for at least about three months, at least six months, at least 9 months, at least 12 months, or at least 18 months. Typically, the reagent preparation is stable over 4° C. In some embodiments, the reagent preparation is stable at room temperature (approximately 20-25° C.).

The primers in the reagent preparation are target-specific. The specific primers are designed so that they permit the amplification of the target nucleotide sequence using the LAMP method. See, e.g., U.S. Pat. No. 6,410,278; U.S. Appl. No. 2006/0141452; and Nagamine et al., Clin. Chem. (2001) 47:1742-43. A primer, which is used for synthesizing the desired nucleic acid sequence, is not particularly limited in length as long as it complementarily binds as necessary. Typically, four or six different primers are employed.

A primer may be bound to, or modified to be bindable to, a detectable label substance or solid phase. When labeling the primer for synthesizing nucleic acid sequences, known substances and methods for labeling can be employed. Examples of label substances include radioactive substances, fluorescent substances, haptens, biotins, and enzymes. These label substances can be added to a primer in accordance with known molecular biology techniques, or a previously labeled nucleotide can be incorporated at the time of chemical synthesis of a primer to prepare a label primer. A suitable functional group may be introduced in the primer so as to be bindable to the aforementioned label substances or latex particles, magnetic particles, or the inner wall of a reaction vessel. The label site of the primer has to be selected in such a manner that annealing to a complementary strand or a subsequent extension reaction is not inhibited. Depending on their molecular weight, label substances can be bound through a base sequence as a linker on the 5′ side to prevent steric hindrance from occurring.

The dinucleotide triphosphates provided in the reagent preparation include dATP, dCTP, dGTP, dTTP, and dUTP as well as useful analogues and derivatives known in the art.

The components of the dry reagent preparation disclosed herein can be at any concentration suitable for the dry process. Usually, the components are at about 5×, 10×, 20× or higher concentration to facilitate drying such that the reaction tube will contain about ⅕, 1/10, 1/20 or less volume than a 1× concentration, where a 1× concentration is the concentration of components used to perform the LAMP method.

The aqueous buffered solution in the additional and separate wet format is one that provides a suitable pH to the to the enzyme reaction, salts necessary for annealing or for maintaining the catalytic activity of the enzyme, a protective agent for the enzyme, and as necessary a regulator for melting temperature (Tₘ). An exemplary buffer is Tris-HCl, having a buffering action in a neutral to weakly alkaline range. The pH is adjusted depending on the DNA polymerase used. As the salts, KCl, NaCl, (NH₄)₂SO₄ etc. are suitably added to maintain the activity of the enzyme and to regulate the melting temperature (Tₘ) of nucleic acid. The protective agent for the enzyme makes use of bovine serum albumin or sugars. Further, dimethyl sulfoxide (DMSO) or formamide can be used as the regulator for melting temperature (Tₘ). By use of the regulator for melting temperature (Tₘ), annealing of the oligonucleotide can be regulated under limited temperature conditions. Further, betaine (N,N,N-trimethylglycine) or a tetraalkyl ammonium salt is also effective for improving the efficiency of strand displacement by virtue of its isostabilization. By adding betaine in an amount of 0.2 to 3.0 M, preferably 0.5 to 1.5 M to the reaction solution, its promoting action on the nucleic acid amplification of the present invention can be expected. Because these regulators for melting temperature act for lowering melting temperature, those conditions giving suitable stringency and reactivity are empirically determined in consideration of the concentration of salts, reaction temperature etc. Thus, in one embodiment, the additional, separate wet format comprises an aqueous buffered solution such as 25 mM Tris-HCl pH 8.8, 12.5 mM KCl, 10 mM MgSO₄, 12.5 mM (NH₄)₂SO₄, and 0.125% Tween 20. In some embodiments, betaine is also included.

Any suitable method of drying can be employed. For example, drying of the disclosed reagent preparation can be effectively performed in a drying chamber such as a lyophilizer. The reagent preparation can be dried in plastic as glass is not required. Also, in some embodiments, the reagent preparation may be frozen prior to drying. For example, product can be dried in plastic microfuge tubes of various sizes and plastic microtiter wells. The dried product is sealed to protect from moisture, e.g., a butyl rubber stopper for a glass tube with the interior chamber similar in shape to a microfuge tube or foil lined plastic pouch or container with desiccant for plastic microfuge tubes and microtiter wells. The length of time of drying varies depending on the method used. A typical drying time is less than 2 hours. After material has reached visible dryness (white pellet) the tube is closed and stored in a desiccated environment to protect product from moisture. In some embodiments, greater than about 90%, sometimes greater than about 95% of the moisture is removed by drying.

The dry and wet format can use any suitable container. Typically, the individual formats are in single, plastic tubes.

Further provided herein is a kit comprising the dry format reagent preparation disclosed herein and a separate, wet format component comprising an aqueous buffered solution suitable for performing the LAMP method on a nucleic acid sample. The kit can be in any suitable physical form and optionally may include instructions.

### EXAMPLE 1

The functionality of the dry format containing the reagents necessary for LAMP were compared. The differences in the format are shown in Table 1.

TABLE 1

| Standard LAMP kit | Dry Format Lamp Kit |
|---|---|
| 2 x Reaction Mix (1 tube) | 1.5 ml Reaction Tube (1 tube) |
| 2M betaine | 32U Bst enzyme |
| 40 mM Tris-Cl pH 8.8 | [and 3U AMV reverse |
| 20 mM KCl | transcriptase (if RNA target)] |
| 20 mM (NH₄)₂SO₄ | Primers (kit dependent) |
| 16 mM MgSO₄ | dNTPs |
| dinucleotide triphosphates (dNTPs) | |
| 0.2% Tween 20 | |
| Primer Mix (1 tube) | Aqueous Buffer (1 tube) |
| Primers (target specific) | 25 mM Tris-HCl pH 8.8 |
| | 12.5 mM KCl |
| | 10 mM MgSO₄ |
| | 12.5 mM (NH₄)₂SO₄ |
| | 0.125% Tween 20 |
| | 1.25 M betaine |
| Enzyme Mix (1 tube) | |
| 8 U/μl Bst polymerase | |
| [and 1 U/μl AMV reverse | |
| transcriptase (if RNA target)] | |
| 50% Glycerol | |
| Distilled Water | Negative Control of |
| (1 tube) | DNAse/RNAse free water |
| | (1 tube) |
| Positive Control | Positive Control |
| (1 tube) | (1 tube) |

Wet Format LAMP. In the standard LAMP kit, the kit components must be stored at −20° C. The recommended protocol is as follows: Remove reagents from −20° C. and thaw at room temperature. Once thawed, keep on ice. Prepare Master Mix (prepare on ice) either in 0.5 ml or 1.5 ml tubes. Briefly, the Master Mix is prepared by adding 12.5 μl 2× Reaction Mix; 2.5 μl Primer Mix; and 4.0 μl distilled water into a reaction tube. Reagents are mixed by tapping or inverting tube or vortex ˜1 second×3 times followed by a brief centrifugation. The tube was heated @ 95° C. for 5 minutes. Then, the tube was cooled on ice. After cooling, 1 μl Enzyme Mix was added to the tube, followed by vortexing and/or centrifuging. Once the Master Mix preparation was complete, 20 μl of Master Mix was dispensed into each sample and control tube (0.2 ml PCR tubes). 5 μl of DNA or RNA sample were added to the tube and mix by pipetting or taping, and then centrifuged briefly. The tubes were heated at ˜60° C. for 1 hour, followed by inactivation of the enzyme at 80° C. for 5 minutes. Turbidity was determined by visual inspection.

Dry Format LAMP. The dry format LAMP reagent preparation greatly reduces the number of steps, thereby reducing errors and increasing sensitivity. The components in the dry format LAMP reagent preparation can be stored at −20° C. to 30° C.

Preparation of dry format. Enzyme-containing solution was dialyzed against enzyme storage buffer that was glycerol-free using a tangential flow microdialyser. Typically, dialysis occurred in less than 2 hours. The dialyzed enzyme solution as well as undialyzed enzyme solution was dried using a lyophilizer. The undialyzed solution was unable to be dried after 24 hours. The tubes containing dried, dialyzed enzyme were stored in a sealed foil pouch containing desiccant.

Protocol. The reaction tube containing the dry reagent preparation was removed the from the foil pouch. 80 μl of the reaction buffer and 20 μl of the sample were added to each reaction tube. The contents were mixed by gently vortexing, and then heat at ˜60° C. for 1 hour. Turbidity was determined visually.

### EXAMPLE 2

The purpose of this experiment was to determine if reverse transcriptase LAMP (RT-LAMP) would function if Bst polymerase and AMV reverse transcriptase were lyophilized in the same tube.

Materials included dNTPS (25 mM) (New England Biolabs); Eiken Norovirus GI primer mix set; dialyzed Bst DNA polymerase (˜37 u/μl, no glycerol); AMV reverse transcriptase (20 u/μl) (Stratagene); AMV dialysis buffer (200 mM KH₂PO₄, 2 mM dithiothreitol (DTT) and 0.2% Triton X-100), pH 7.2; reconstitution buffer (2×): 40 mM Tris-HCl pH 8.8, mM KCl, 16 mM MgSO₄, 20 mM (NH₄)₂SO₄, and 0.2% Tween 20; and betaine.

Procedure—Lyophilization of Enzyme Mix

| 1. Prepared enzyme dilutions | | |
|---|---|---|
| a. | Bst 8 u/μl: | 2.4 μl dialyzed enzyme + |
| | | 7.6 μl dH₂O |
| b. | AMV 0.5 u/μl: | 0.7 μl dialyzed enzyme + |
| | | 9.3 μl dH₂O |
| 2. Prepare enzyme mix in three 0.2 ml tubes | | |
| a. | Norovirus GI primer mix: | 2.5 μl per tube |
| b. | Diluted Bst | 1.0 μl per tube |
| c. | Diluted AMV | 1.0 μl per tube |
| d. | 25 mM dNTPs | 1.4 μl per tube |
| 3. Enzyme mix lyophilized 30 minutes. | | |
| 4. Added reconstitution buffer components to reaction tube | | |
| a. | 2X reaction buffer | 12.5 μl/tube |
| b. | Betaine | 4.0 μl/tube |
| c. | dH₂O | 3.5 μl/tube |
| d. | Norovirus GI RNA or dH₂O | 5.0 μl/tube (1 positive/ |
| | | 2 negative) |

5. Incubated at 63° C. for 60 minutes
6. Results interpreted visually.

Results—Lamp Reaction with Lyophilized Reagents:

Norovirus GI positive control: (+)

Water (negative control): (−), (−)

Conclusions: Reverse transcriptase LAMP can successfully be performed with AMV reverse transcriptase and Bst enzyme lyophilized in the same tube.

### EXAMPLE 3

Purpose: The purpose of this experiment was to confirm the requirement to remove glycerol from the enzyme storage buffer prior to lyophilization.

Materials included dNTPS (25 mM) (New England Biolabs); Clostridium difficile TcdB (Toxin B) Loopamp primer set; Bst DNA polymerase (120 u/μl) (New England Biolabs); Bst DNA polymerase (8 u/μl) (New England Biolabs); and Bst DNA dialysis buffer (50 mM KCl, 10 mM Tris-HCl pH 7.5, 0.1 mM EDTA, 1 mM dithiothreitol (DTT) and 0.1% Triton X-100), pH 7.5.

Procedure—Lyophilization of Enzyme Mix: 10 reactions tubes each were prepared for the undialyzed and dialyzed enzyme by preparing a 10.5 reaction volume for each enzyme condition in one tube and aliquoting single reaction volume into 10 tubes as follows:

| | 10.5 volume | per reaction tube |
|---|---|---|
| Undialyzed: | | |
| dNTP | 58.8 μl | 5.6 μl |
| Primer mix | 42 μl | 4.0 μl |
| Bst (8 u/μl) | 84 μl | 8.0 μl |
| Dialyzed: | | |
| dNTP | 58.8 μl | 5.6 μl |
| Primer mix | 42 μl | 4.0 μl |
| Bst (37 u/μl) | 21 μl | 2.0 μl |

Lyophilization monitored through glass at 8 minutes, 30 minutes, 45 minutes, 60 minutes, 120 minutes and 27.5 hours (for undialyzed enzyme reagent tubes only). Reaction tubes containing dialyzed enzyme were removed after 2 hours of lyophilization. Reaction tubes containing undialyzed enzyme were removed after 27.5 hours of lyophilization.

Results—Lyophilization of Enzyme Mix. All ten reagent tubes containing dialyzed enzyme appeared to be visually dry at 8 minutes. Reagent confirmed to be dry after 2 hours of lyophilization. (“Dry” is defined as material that has transitioned from a clear liquid to a white “fluffy” solid). All tubes containing undialyzed enzyme did not appear visually dry at any time during lyophilization however at 45 minutes, a visually noticeable decrease in volume was observed. All tubes containing undialyzed enzyme still appeared wet and clear after 27.5 hours of lyophilization.

Conclusion: The glycerol supplied with the Bst enzyme must be removed prior to lyophilization for the product to form a dry reagent.

1. A reagent preparation for loop-mediated isothermal amplification of nucleic acids comprising:
at least one polymerase enzyme,
a target-specific primer set, and
dinucleotide triphosphates (dNTPs) in a single, dry format;
wherein said reagent preparation is water soluble and stable above 4° C.

2. The reagent preparation of claim 1, wherein said polymerase enzyme is Bst enzyme.

3. The reagent preparation of claim 1, further comprising a reverse transcriptase.

4. The reagent preparation of claim 3, wherein said reverse transcriptase is AMV reverse transcriptase.

5. A kit comprising the reagent preparation of claim 1.

6. The kit of claim 5, further comprising a separate wet format comprising an aqueous buffered solution.

7. The kit of claim 5, wherein said solution is 25 mM Tris-HCl pH 8.8, 12.5 mM KCl, 10 mM MgSO₄, 12.5 mM (NH₄)₂SO₄, and 0.125% Tween 20.

8. A method of making a reagent preparation for loop-mediated isothermal amplification of nucleic acids comprising the steps of:
(a) providing a buffered aqueous solution of
(1) at least one polymerase enzyme,
(2) a target-specific primer set,
(3) dinucleotide triphosphates (dNTPs),
wherein said solution is glycerol-free; and
(b) drying the solution to form the reagent preparation;
wherein the reagent preparation is water soluble and is stable above 4° C.

9. The method of claim 8, wherein said polymerase enzyme is thermostable.

10. The method of claim 8, further comprising a reverse transcriptase.

11. The method of claim 10, wherein said reverse transcriptase is AMV reverse transcriptase.
